(19) United States
(12) Patent Application Publication
(10) Pub. No.: US 2022/0034766 A1
(43) Pub. Date: Feb. 3, 2022

(54) IMMUNOHISTOCHEMICAL STAINING FOR GSK-3

(71) Applicant: Actuate Therapeutics, Inc., Fort Wortha, TX (US)

(72) Inventor: Andrey UGOLKOV, Vernon Hills, IL (US)

(21) Appl. No.: 17/312,590

(22) PCT Filed: Dec. 12, 2019
(86) PCT No.: PCT/US2019/065992

§ 371 Date: Jun. 10, 2021

Related U.S. Application Data
(60) Provisional application No. 62/778,655, filed on Dec. 12, 2018.

(51) Int. Cl. G01N 1/30; G01N 33/574

(52) U.S. Cl. CPC G01N 1/30; G01N 33/574; G01N 2800/7028; G01N 2800/52; G01N 2001/302

(57) ABSTRACT

Methods of using immunohistochemical staining to identify cells expressing GSK-3 and use of such methods in treating disease are disclosed.

### CROSS-REFERNCE TO RELATED APPLICATION

This application claims the benefit of priority from U.S. Provisional Application No. 62/778,655, filed Dec. 12, 2018, the entirety of which is incorporated by reference herein.

### TECHNICAL FIELD

The present disclosure relates to methods of using immunohistochemical staining to identify tumor cells expressing GSK-3β or GSK-3α and use of such methods to identify cancers susceptible to treatment with inhibitors specific to these targets.

### BACKGROUND

A biomarker is a “a characteristic that is objectively measured and evaluated as an indicator of normal biological processes, pathogenic processes, or pharmacologic responses to a therapeutic intervention.” Kyle Strimbu and Jorge A. Tavel, What are Biomarkers?, Curr Opin HIV AIDS 2010 November 5(6): 463-466. Thus, biomarkers can identify patients in whom pathogenic processes are underway, and in doing so identify patients for whom a given therapeutic regimen is likely to be effective.

Glycogen synthase kinase-3 (GSK-3) is a serine (S)/threonine (T) kinase initially described as a key regulator of metabolism, specifically glycogen biosynthesis. Embi N, et al. Glycogen synthase kinase-3 from rabbit skeletal muscle. Separation from cyclic-AMPdependent protein kinase and phosphorylase kinase. Eur J Biochem. 1980; 107:519-27. GSK-3 has since been shown to play a role in several disease processes, including cancer and aging, immune disorders, metabolic disorders, and neurological disorders through modulation of a large and diverse number of substrates. Sutherland C. What Are the bona fide GSK3 Substrates? Int J Alzheimers Dis. 2011; 2011:505607; Gao C, et al. GSK3: a key target for the development of novel treatments for type 2 diabetes mellitus and Alzheimer disease. Rev Neurosci. 2011; 23:1-11; Wang H, et al., Convergence of the mammalian target of rapamycin complex 1- and glycogen synthase kinase 3-beta-signaling pathways regulates the innate inflammatory response. J Immunol. 2011; 186:5217-26; Klamer G, et al. Using small molecule GSK3beta inhibitors to treat inflammation. Curr Med Chem. 2010; 17:2873-81; Henriksen E J. Dysregulation of glycogen synthase kinase-3 in skeletal muscle and the etiology of insulin resistance and type 2 diabetes. Curr Diabetes Rev. 2010; 6:285-93.

GSK-3 has two ubiquitously expressed and highly conserved isoforms, GSK-3α and GSK-3β, with both shared and distinct substrates and functional effects. Aberrant overexpression of GSK-3α or GSK-3β has been shown to promote tumor growth and chemotherapy resistance in various malignant tumors.

GSK-3β inhibitors are of interest due to their ability to potentially alter the clinical course of diseases mediated by GSK-3β. Some GSK-3β inhibitors include tideglusib (4-benzyl-2-(naphthalen-1-yl)-[1,2,4]thiadiazolidine-3,5-dione), LY2090314 (3-[9-fluoro-2-(piperidin-1-ylcarbonyl)-1,2,3,4-tetrahydro[1,4]diazepino[6,7,1-hi]indol-7-yl]-4-imidazo[1,2-a]pyridin-3-yl-1H-pyrrole-2,5-dione), 9-ING-41 (3-(5-fluorobenzofuran-3-yl)-4-(5-methyl-5H-[1,3]dioxolo[4,5-f]indol-7-yl)pyrrole-2,5-dione), CHIR-99021(6-((2-((4-(2,4-dichlorophenyl)-5-(5-methyl-1H-imidazol-2-yl)pyrimidin-2-yl)amino)ethyl)amino)nicotinonitrile) and CHIR-98014 (N2-(2-(4-(2,4-dichlorophenyl)-5-(1H-imidazol-1-yl)pyrimidin-2-ylamino)ethyl)-5-nitropyridine-2,6-diamine), SB216763 (3-(2,4-dichlorophenyl)-4-(1-methyl-1H-indol-3-yl)-1H-pyrrole-2,5-dione) and SB415286 (3-(3-chloro-4-hydroxyphenylamino)-4-(2-nitrophenyl)-1H-pyrrole-2,5-dione), AR-A014418 (N-[(4-Methoxypheny)methyl]-N′-(5-nitro-2-thiazoiyl)urea), CG701338 and CG202796. See Amy Walz, Andrey Ugolkov, Sunandana Chandra, et al., Molecular Pathways: Revisiting Glycogen Synthase Kinase-3β as a Target for the Treatment of Cancer, Clin Cancer Res; 23(8) Apr. 15, 2017, OF1-OF7.

A need exists for methods of identifying tumor cells that are expressing one or more GSK-3 isoforms so that inhibitors of specific GSK-3 isoforms may be administered to such patients. Moreover, previous methods for nuclear counterstaining of immunohistochemical samples require up to an hour of contacting the sample with a nuclear counterstain such as hematoxylin. A need therefore exists for more rapid and efficient methods of identifying tumor cells in biological samples.

### SUMMARY

In some aspects, the present disclosure is directed to a method of treating disease in a subject in need thereof by a) obtaining a tissue sample from the subject; b) measuring the expression level of a GSK-3 isoform in the sample, wherein the measuring is performed by immunohistochemical staining; c) comparing the expression level of the GSK-3 isoform in the tissue sample with the expression level of that GSK-3 isoform in a control sample; and d) administering an effective amount of an inhibitor of the GSK-3 isoform to the subject if an elevated expression level of the GSK-3 isoform in the tissue sample is detected when compared to the expression level of the GSK-3 isoform in the control sample.

In some aspects, the present disclosure is directed to a method of the preceding aspect wherein the expression level of GSK-3 isoform is nuclear expression.

In some aspects, the present disclosure is directed to a method of the preceding aspects wherein the expression level of GSK-3 isoform is cytoplasmic expression.

In some aspects, the present disclosure is directed to a method of the preceding aspects wherein the expression level of GSK-3 isoform is nuclear and cytoplasmic expression.

In some aspects, the present disclosure is directed to a method of the preceding aspects wherein the GSK-3 isoform is GSK-3β.

In some aspects, the present disclosure is directed to a method of the preceding aspects wherein the inhibitor of the GSK-3 isoform is 9-ING-41, tideglusib, or LY2090314.

In some aspects, the present disclosure is directed to a method of the preceding aspects wherein the inhibitor of the GSK-3 isoform is 9-ING-41.

In some aspects, the present disclosure is directed to a method of the preceding aspects wherein the GSK-3 isoform is GSK-3α.

In some aspects, the present disclosure is directed to a method of the preceding aspects wherein the disease is cancer.

In some aspects, the present disclosure is directed to a method of the preceding aspects wherein the tissue sample is a tumor sample.

In some aspects, the present disclosure is directed to a method of the preceding aspects wherein the cancer is breast cancer, brain cancer, esophagus cancer, stomach cancer, lung cancer, liver cancer, kidney cancer, thyroid gland cancer, spleen cancer, pancreas cancer, large bowel cancer, skin cancer, ovarian cancer, uterus cancer, prostate cancer, bladder cancer, testicular cancer, osteosarcoma, fibrosarcoma.

In some aspects, the present disclosure is directed to a method of the preceding aspects wherein the disease is a malignant lymphoproliferative disorder.

In some aspects, the present disclosure is directed to a method of the preceding aspects wherein the disease is pulmonary fibrosis or pleural fibrosis.

In other aspects, the present disclosure is directed to an immunohistochemical method for detecting elevated expression of a GSK-3 isoform in a biological sample from an individual through an antigen-antibody reaction comprising, (1) contacting said biological sample with a first antibody specific for a GSK-3 isoform to give an antibody-contacted biological sample; (2) contacting said first antibody-contacted biological sample with a secondary antibody specific for said first antibody, wherein said secondary antibody has an enzymatic activity; (3) treating the product of (2) with a chromogenic substrate for said enzymatic activity; and (4) counterstaining the product of (3) with a counterstain for up to 8 minutes.

In some aspects, the present disclosure is directed to a method of the preceding aspect wherein the counterstain is hematoxylin.

In some aspects, the present disclosure is directed to a method of the preceding aspect wherein the counterstaining with hematoxylin is performed for up to 4 minutes.

In some aspects, the present disclosure is directed to a method of the preceding aspects wherein the counterstaining with hematoxylin is performed for up to 2 minutes.

In some aspects, the present disclosure is directed to a method of the preceding aspects wherein the counterstaining with hematoxylin is performed for up to 1 minute.

In some aspects, the present disclosure is directed to a method of the preceding aspects wherein the counterstaining with hematoxylin is performed for up to 30 seconds.

In some aspects, the present disclosure is directed to a method of the preceding aspects wherein the counterstaining with hematoxylin is performed for up to 10 seconds.

In some aspects, the present disclosure is directed to a method of the preceding aspects wherein the counterstaining with hematoxylin is performed for between 10 seconds and 30 seconds.

In some aspects, the present disclosure is directed to a method of the preceding aspects wherein the enzymatic activity of the secondary antibody is peroxidase activity.

In some aspects, the present disclosure is directed to a method of the preceding aspects wherein the enzymatic activity of the secondary antibody is alkaline phosphatase activity.

In some aspects, the present disclosure is directed to a method of the preceding aspects wherein the chromogenic substrate is a peroxidase substrate.

In some aspects, the present disclosure is directed to a method of the preceding aspects wherein the chromogenic substrate is an alkaline phosphatase substrate.

In some aspects, the present disclosure is directed to a method of the preceding aspects wherein the first antibody is a monoclonal antibody.

In some aspects, the present disclosure is directed to a method of the preceding aspects wherein the second antibody is a monoclonal antibody.

In some aspects, the present disclosure is directed to kits for use in immunohistochemical staining for GSK-3, wherein said kit comprises
(a) a GSK-3 inhibitor;
(b) a GSK-3 specific antibody;
(c) a secondary antibody that is specific for the GSK-3 specific antibody, wherein the secondary antibody also has a conjugated enzymatic activity;
(d) a chromogenic substrate for the enzymatic activity of the secondary antibody;
(e) a counterstain;
(f) and instructions for use of the kit.

In some aspects, the present disclosure is directed to kits of the preceding aspect, wherein the GSK-3 inhibitor is 9-ING-41, tideglusib, LY2090314, CHIR-99021, CHIR-98014, SB216763, SB415286, AR-A014418, CG701338, or CG202796

In some aspects, the present disclosure is directed to kits of the preceding aspects, wherein the GSK-3 inhibitor is 9-ING-41.

In some aspects, the present disclosure is directed to kits of the preceding aspects, wherein the GSK-3 specific antibody is a GSK-3beta specific monoclonal antibody.

In some aspects, the present disclosure is directed to kits of the preceding aspects, wherein conjugated enzymatic activity is peroxidase activity or alkaline phosphatase activity.

In some aspects, the present disclosure is directed to kits of the preceding aspects, wherein conjugated enzymatic activity is peroxidase activity.

In some aspects, the present disclosure is directed to kits of the preceding aspects, wherein chromogenic substrate is diaminobenzidine (DAB), 3-Amino-9-ethylcarbazole (AEC), 5-bromo-4-chloro-3-indolyl phosphate/tetranitroblue tetrazolium (BCIP/TNBT), or Naphthol AS-MX phosphate+Fast Blue BB.

In some aspects, the present disclosure is directed to kits of the preceding aspects, wherein chromogenic substrate is diaminobenzidine (DAB).

In some aspects, the present disclosure is directed to kits of the preceding aspects, wherein the counterstain is hematoxylin or methyl green.

In some aspects, the present disclosure is directed to kits of the preceding aspects, wherein the counterstain is hematoxylin.

In some aspects, the present disclosure is directed to kits of the preceding aspects, wherein for use in the method of any of the aspects of the disclosure.

### BRIEF DESCRIPTION OF THE DRAWINGS

FIG. 1 shows GSK-3α and GSK-3β immunohistochemical staining of breast cancer (ductal adenocarcinoma). (Darkly shaded regions indicate positive expression of GSK-3β or GSK-3α in cancer cells; see, e.g., arrows.)

FIG. 2 shows GSK-3α and GSK-3β immunohistochemical staining of colon cancer. (Darkly shaded regions indicate cytoplasmic expression of GSK-3β or GSK-3α in cancer cells; see, e.g., arrows.)

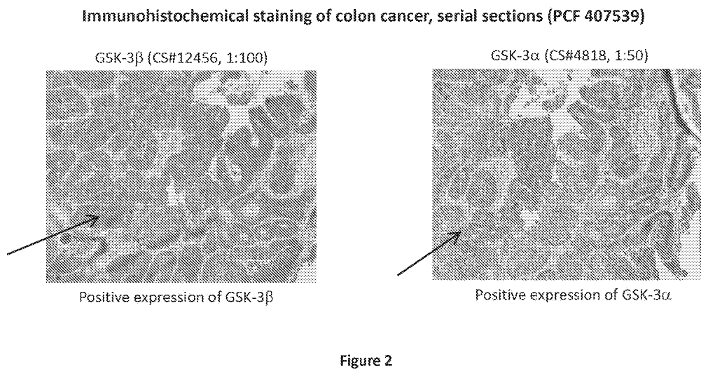

FIG. 3 shows immunohistochemical staining of breast cancer (ductal adenocarcinoma) for GSK-3β (1:100) with hematoxylin counter-staining for 30 seconds. (Darkly shaded regions indicate nuclear expression of GSK-3β; see, e.g., arrow.)

FIG. 4 shows immunohistochemical staining of breast cancer (ductal adenocarcinoma) for GSK-3β (1:100) with hematoxylin counter-staining for 30 seconds. (Darkly shaded regions indicate nuclear expression of GSK-3β in cancer cells; see, e.g., arrow.)

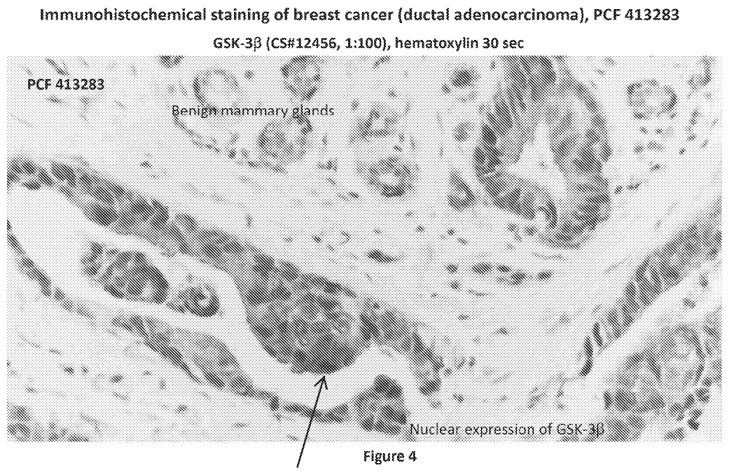

### DETAILED DESCRIPTION OF ILLUSTRATIVE EMBODIMENTS

The present subject matter may be understood more readily by reference to the following detailed description which forms a part of this disclosure. It is to be understood that this invention is not limited to the specific methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treating”, “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. This condition, disease or disorder can be cancer.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with the particular compound, component or composition selected, the route of administration, and the ability of the components to elicit a desired result in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide the improved therapeutic response. An effective amount is also one in which any toxic or detrimental effects of the components are outweighed by the therapeutically beneficial effects.

As employed above and throughout the disclosure the term “sub-therapeutic amount” refers to an amount that is ineffective when administered as the sole therapeutic agent.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Within the present invention, the disclosed compounds may be prepared in the form of pharmaceutically acceptable salts. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein can be prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxy groups often exist in equilibrium with their zwitterionic forms. Thus, any of the compounds described herein that contain, for example, both amino and carboxy groups, also include reference to their corresponding zwitterions.

The term “administering” means either directly administering a compound or composition of the present invention, or administering a prodrug, derivative or analog which will form an equivalent amount of the active compound or substance within the body.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys.

In some aspects, the present disclosure is directed to a method of treating disease in a subject in need thereof by a) obtaining a tissue sample from the subject; b) measuring the expression level of a GSK-3 isoform in the sample, wherein the measuring is performed by immunohistochemical staining; and c) comparing the expression level of the GSK-3 isoform in the tissue sample with the expression level of that GSK-3 isoform in a control sample; d) administering an effective amount of an inhibitor of the GSK-3 isoform to the subject if an elevated expression level of the GSK-3 isoform in the tissue sample is detected when compared to the expression level of the GSK-3 isoform in the control sample.

The diseases that may be treated using the methods of the present disclosure include any diseases or conditions which are characterized by elevated expression of a GSK-3 isoform. Such diseases include cancers, such as, for example, breast cancer, brain cancer, esophagus cancer, stomach cancer, lung cancer, liver cancer, kidney cancer, thyroid gland cancer, spleen cancer, pancreatic cancer, colon cancer, skin cancer, ovarian cancer, uterus cancer, prostate cancer, bladder cancer, testicular cancer, osteosarcoma, or fibrosarcoma. In some embodiments, the cancer is breast cancer, pancreatic cancer, or colon cancer. In some embodiments, the cancer is breast cancer. In other embodiments, the cancer is pancreatic cancer. In yet other embodiments, the cancer is colon cancer.

Other diseases that may be treated using the methods of the present disclosure include malignant lymphoproliferative disorders. Such disorders include Malignant B-cell lymphoproliferative disorders and Malignant T-cell lymphoproliferative disorders. Malignant B-cell lymphoproliferative disorders include Diffuse large B-cell lymphoma, acute lymphocytic leukemia, lymphoid blastic phase Chronic Myeloid Leukemia, Chronic lymphocytic leukemia/Small lymphocytic lymphoma, Extranodal marginal zone B-cell lymphomas, Mucosa-associated lymphoid tissue lymphomas, Follicular lymphoma, Mantle cell lymphoma, Nodal marginal zone B-cell lymphoma, Burkitt lymphoma, Hairy cell leukemia, Primary central nervous system lymphoma, Splenic marginal zone B-cell lymphoma, Waldenstrom's macroglobulinemia/Lymphoplasmacytic lymphoma, Multiple myeloma, Plasma cells dyscrasias, Plasma cell neoplasms, Primary mediastinal B-cell lymphoma, Hodgkin Disease, and Castelman's Disease. Malignant T-cell lymphoproliferative disorders include T-cell leukemia/lymphoma, Extranodal natural killer/T-cell lymphoma, Cutaneous T-cell lymphoma, Enteropathy-type T-cell lymphoma, Angioimmunoblastic T-cell lymphoma, Anaplastic large T/null-cell lymphoma, Subcutaneous panniculitis-like T-cell lymphoma, T-cell acute lymphocytic leukemia, T-cell large granular lymphocyte leukemia, Lymphoid blastic phase Chronic Myeloid Leukemia, post-transplantation lymphoproliferative syndromes, human T-cell leukemia virus type 1-positive (HTLV-1⁺) adult T-cell leukemia/lymphoma (ATL), T-cell prolymphocytic leukemia (T-PLL), and unspecified T-cell lymphoma.

Other diseases that may be treated using the methods of the present disclosure include pulmonary fibrosis or pleural fibrosis.

The methods of the present disclosure are practiced on a subject in need thereof. In some embodiments, the subject in need thereof is an animal. In other embodiments, the subject in need thereof is a mammal. In yet other embodiments, the subject in need thereof is a dog or a cat. In other embodiments, the subject in need thereof is s human.

In the methods of treatment of the present disclosure, a tissue sample from the subject is obtained. The tissue sample may be any specimen or collection of cells of a tissue that is affected by the disease or condition which is to be treated. Where the disease being treated is a cancer, for example, the tissue sample is a sample of the tumor. Where the disease being treated is a malignant lymphoproliferative disorder, the sample is a sample of the blood cells. Where that disease is pulmonary or pleural fibrosis, the tissue is a sample of pleural tissue or lung tissue.

In the methods of the present disclosure, the expression level of a GSK-3 isoform in the sample is determined by immunohistochemical staining. Methods of performing immunohistochemical staining are generally known by those of skill in the art. In brief, the tissue sample is contacted with a GSK-3 isoform-specific antibody. After an incubation period, the tissue sample is contacted with a secondary antibody. The secondary antibody recognizes and binds to the first antibody. The secondary antibody may contain a conjugated activity (e.g., an enzymatic activity) that is used to detect the presence of the secondary antibody, and thus the presence of the first antibody, and thus the presence of the GSK-3 isoform. Example conjugated activities can be any known to those skilled in the art to be useful for creating a detectable immunohistochemical signal. Suitable enzyme conjugates for the secondary antibody include, for example, horseradish peroxidase (HRP), alkaline phosphatase, glucose oxidase, and β-galactosidase; also contemplated are fluorescent probes, radioactive isotopes, chemiluminescent compounds, bioluminescent compounds, or combinations thereof.

In some embodiments, the GSK-3 isoform-specific antibody is specific for GSK-3β. In some embodiments, the GSK-3β-specific antibody is a commercially-available GSK-3β antibody. In some embodiments, the GSK-3β-specific antibody is a polyclonal antibody. In some embodiments, the GSK-3β-specific antibody is a monoclonal antibody. In some embodiments, the GSK-3β-specific antibody is a rabbit antibody. In some embodiments, the GSK-3β-specific antibody is GSK-3beta (D5C5Z) XP rabbit mAb #12456, from Cell Signaling Technology.

In other embodiments, the GSK-3 isoform-specific antibody is specific for GSK-3α. In some embodiments, the GSK-3α-specific antibody is a commercially-available GSK-3α antibody. In some embodiments, the GSK-3α-specific antibody is a polyclonal antibody. In some embodiments, the GSK-3α-specific antibody is a monoclonal antibody. In some embodiments, the GSK-3α-specific antibody is a rabbit antibody. In some embodiments, the GSK-3α-specific antibody is GSK-3alpha (D80D1) XP rabbit mAb #4818, from Cell Signaling Technology.

In some embodiments, the GSK-3 isoform-specific antibody is specific for a GSK-3 isoform in a predetermined phosphorylation state of the GSK-3 isoform. For example, the GSK-3 isoform-specific antibody can be specific for a phosphorylated form of the GSK-3 isoform, or the GSK-3 isoform-specific antibody can be specific for a non-phosphorylated form of the GSK-3 isoform.

In some embodiments, the secondary antibody is commercially available. In some embodiments, the secondary antibody is a Peroxidase labelled polymer conjugated to goat anti-rabbit immunoglobulins, such as that contained in EnVision+System-HRP kit (DAKO, Carpinteria, Calif.).

In the methods of the present disclosure, the GSK-3 isoform expression to be measured may be nuclear GSK-3, cytoplasmic GSK-3, or both nuclear and cytoplasmic GSK-3. In some embodiments, the nuclear expression level of GSK-3 isoform is determined. In other embodiments, the cytoplasmic expression level of GSK-3 isoform is determined. In some embodiments, the nuclear and cytoplasmic expression level of GSK-3 isoform is determined.

In the methods of the present disclosure, after the level of expression of the GSK-3 isoform in the tissue sample is determined, that level is compared to the expression level of the same GSK-3 isoform in a control sample. In some embodiments, the control sample is of the same or comparable tissue type as the tissue sample, but is known to have normal expression levels of the GSK-3 isoform. In some embodiments, the control sample is normal, or non-diseased tissue of the same tissue type as the tissue sample, but taken from an individual or group of individuals known to exhibit normal expression levels of the GSK-3 isoform. In some embodiments, the control sample is normal, or non-diseased tissue of the same or comparable tissue type as the tissue sample taken from the same individual as the tissue sample. In some embodiments, the control sample comprises a normal, or non-diseased sub-population of cells within the tissue sample. Normal, or non-diseased, as used herein, refers to cells or tissue that does not exhibit the phenotype of elevated level of GSK-3 isoform expression.

In the methods of the present disclosure, if an elevated expression level of the GSK-3 isoform in the tissue sample as compared to the expression level of the GSK-3 isoform in a control sample is detected, then an inhibitor of the GSK-3 isoform is administered to the subject with elevated expression level of the GSK-3 isoform. An elevated expression level will have been detected when the GSK-3 isoform expression level in the tissue sample is higher than the GSK-3 isoform expression level in the control sample. GSK-3β is not expressed in benign cells or tissue (served as internal negative control) adjacent to cancer cells within a malignant tumor. Thus higher expression of GSK-3β in cancer cells is determined by visual comparison of cancer cells to adjacent benign cells at the same tumor specimen. Positive expression of GSK-3β can be defined as cytoplasmic and/or nuclear positive staining of more than 50% of cancer cells.

In the methods of the present disclosure, an inhibitor of the GSK-3 isoform is administered to a subject whose tissue sample is determined to contain an elevated level of the GSK-3 isoform relative to a control sample. In some embodiments, the subject is administered a GSK-3 inhibitor. Exemplary GSK-3inhibitors include 9-ING-41, tideglusib, LY2090314, CHIR-99021, CHIR-98014, SB216763, SB415286, AR-A014418, CG701338, or CG202796. In some embodiments, the subject is administered 9-ING-41, tideglusib, or LY2090314. In some embodiments, the subject is administered 9-ING-41. In some embodiments, the subject is administered tideglusib. In some embodiments, the subject is administered LY2090314.

In some embodiments, the methods of the present disclosure further comprise administering a GSK-3 inhibitor in combination with one or more other therapeutic agents. When the GSK-3 inhibitor is administered in combination with a second therapeutic agent, the treatment is referred to as combination therapy. In combination therapy, it is not necessary that the GSK-3 inhibitor and the second therapeutic agent be introduced into, or applied onto, the patient's body simultaneously. Combination therapy requires only that the GSK-3 inhibitor and the second therapeutic agent be present in or on the patient's body at the same time. Thus, combination therapy does not imply any particular dosing schedule.

In some embodiments, the GSK-3 inhibitor is administered in combination with a second therapeutic agent, wherein the second therapeutic agent is administered in a sub-therapeutic amount.

In some embodiments, the GSK-3 inhibitor is administered in combination with a second therapeutic agent, wherein the second therapeutic agent is an anticancer agent.

In some embodiments of the combination therapy of the present disclosure, the GSK-3 inhibitor is a GSK-3β inhibitor. In other embodiments of the combination therapy, the GSK-3 inhibitor is a GSK-3α inhibitor.

In still other embodiments, the second therapeutic agent is one or more of ABT-737, BAY-1143572, 5-fluorouracil, abiraterone acetate, acetylcholine, ado-trastuzumab emtansine, afatinib, aldesleukin, alectinib, alemtuzumab, alitretinoin, aminolevulinic acid, anastrozole, anastrozole, aprepitant, arsenic trioxide, asparaginase erwinia chrysanthemi, atezolizumab, axitinib, azacitidine, belinostat, bendamustine, benzyl isothiocyanate, bevacizumab, bexarotene, bicalutamide, bleomycin, blinatumomab, bortezomib, bosutinib, brentuximab vedotin, busulfan, cabazitaxel, cabozantinib, capecitabine, carboplatin, carfilzomib, carmustine, ceritinib, cetuximab, chlorambucil, cisplatin, clofarabine, cobimetinib, copanlisib crizotinib, cyclophosphamide, cytarabine, dabrafenib, dacarbazine, dacarbazine, dactinomycin, daratumumab, dasatinib, daunorubicin, decitabine, defibrotide sodium, degarelix, denileukin diftitox, denosumab, dexamethasone, dexrazoxane, dihydrotestosterone (DHT), dinutuximab, docetaxel, doxorubicin, elotuzumab, eltrombopag, enzalutamide, epirubicin, eribulin mesylate, erlotinib, etoposide, everolimus, exemestane, exemestane, filgrastim, fludarabine phosphate, flutamide, fulvestrant, fulvestrant, gefitinib, gemcitabine, gemtuzumab, gemtuzumab ozogamicin, glucarpidase, goserelin acetate, hydroxyurea, ibritumomab tiuxetan, ibrutinib, idarubicin, idelalisib, ifosfamide, imatinib, imiquimod, interferon alfa-2b, ipilimumab, irinotecan, ixabepilone, ixazomib, lanreotide, lapatinib, lenalidomide, lenvatinib, letrozole, leucovorin, leuprolide, lomustine, mechlorethamine, megestrol acetate, melphalan, mercaptopurine, mesna, methotrexate, mitomycin C, mitoxantrone, navitoclax, necitumumab, nelarabine, netupitant, nilotinib, nilutamide, nivolumab, obinutuzumab, ofatumumab, olaparib, omacetaxine mepesuccinate, osimertinib, oxaliplatin, ozogamicin, paclitaxel, palbociclib, palifermin, pamidronate, panitumumab, panobinostat, pazopanib, pegaspargase, peginterferon alfa-2b, pembrolizumab, pemetrexed, pertuzumab, plerixafor, pomalidomide, ponatinib, pralatrexate, prednisone, procarbazine, propranolol, radium 223 dichloride, raloxifene, ramucirumab, rasburicase, regorafenib, rituximab, rolapitant, romidepsin, romiplostim, ruxolitinib, siltuximab, sipuleucel-t, sonidegib, sorafenib, sunitinib, talimogene laherparepvec, tamoxifen, temozolomide, temsirolimus, thalidomide, thioguanine, thiotepa, tipiracil, topotecan, toremifene, toremifene, tositumomab, trabectedin, trametinib, trastuzumab, tretinoin, trifluridine, uridine triacetate, vandetanib, vemurafenib, venetoclax, vinblastine, vincristine, vinorelbine, vismodegib, vorinostat, ziv-aflibercept, zoledronic acid, and pharmaceutically acceptable salts thereof. In some embodiments, the second therapeutic agent is one or more of rituximab, cyclophosphamide, doxorubicin, vincristine, and prednisone.

The present disclosure is also directed to a novel immunohistochemical method for detecting elevated expression of a GSK-3 isoform in an individual through an antigen-antibody reaction comprising, (1) obtaining a biological sample; (2) contacting said biological sample with a first antibody specific for a GSK-3 isoform; (3) contacting the biological sample with a secondary antibody specific for the first antibody, wherein the secondary antibody has enzymatic activity; (4) contacting the product of (3) with a chromogenic substrate for said enzymatic activity; and (5) counterstaining the product of (4) with a counterstain for up to 8 minutes.

In the immunohistochemical staining method of the present disclosure, a biological sample is obtained. The biological sample may be any specimen of tissue or any collection of cells from a tissue. The biological sample may come from any animal or human being. In some embodiments, the biological sample is from a human being. In other embodiments, the biological sample is from an animal.

In the immunohistochemical staining method of the present disclosure, the biological sample is contacted with a first antibody specific for a GSK-3 isoform to give a primary antibody-contacted biological sample. The first antibody is specific for a GSK-3 isoform, meaning that the antibody selectively binds to that specific GSK-3 isoform. In some embodiments, the first antibody is specific for GSK-3β. In other embodiments, the first antibody is specific for GSK-3α. In some embodiments, the first antibody is a polyclonal antibody. In some embodiments, the first antibody is a monoclonal antibody. In some embodiments, the first antibody is a rabbit polyclonal antibody. In some embodiments, the first antibody is a rabbit monoclonal antibody.

In some embodiments, the first antibody is a GSK-3 isoform-specific antibody that is specific for a GSK-3 isoform in a predetermined phosphorylation state of the GSK-3 isoform. For example, the GSK-3 isoform-specific antibody can be specific for a phosphorylated form of the GSK-3 isoform, or the GSK-3 isoform-specific antibody can be specific for a non-phosphorylated form of the GSK-3 isoform.

In the immunohistochemical staining method of the present disclosure, the first antibody-contacted biological sample is contacted with a secondary antibody that is specific for the first antibody, wherein the secondary antibody also has a conjugated activity. The secondary antibody must bind selectively to the first antibody. The secondary antibody can be from the same species as the first antibody, or from a different species than the first antibody. The secondary antibody can be a polyclonal antibody or a monoclonal antibody.

The secondary antibody also has a conjugated activity, which can be an enzymatic activity. In some embodiments, the enzymatic activity is an inherent activity of the secondary antibody. In other embodiments, the enzymatic activity of the secondary antibody is provided by an enzyme that is conjugated to the antibody.

In some embodiments, the enzymatic activity of the secondary antibody is peroxidase activity. In other embodiments, the enzymatic activity of the secondary antibody is alkaline phosphatase activity. Exemplary conjugated enzymatic activities can be any known to those skilled in the art to be useful for creating a detectable immunohistochemical signal, including, for example, horseradish peroxidase (HRP), alkaline phosphatase, glucose oxidase, and β-galactosidase. Other immunohistochemical signals are also contemplated, including, for example, fluorescent probes, radioactive isotopes, chemiluminescent compounds, bioluminescent compounds, or combinations thereof

In the immunohistochemical staining method of the present disclosure, the product of contacting the first antibody-contacted biological sample with a secondary antibody is a biological sample to which is bound the first antibody, and wherein the secondary antibody is bound to the first antibody. In the methods of the present disclosure, this product is contacted with a chromogenic substrate for the enzymatic activity of the secondary antibody.

The chromogenic substrate for the enzymatic activity of the secondary antibody is a chemical compound that changes color upon being reacted with the enzymatic activity of the secondary antibody. In some embodiments, the chromogenic substrate is diaminobenzidine (DAB). In other embodiments, the chromogenic substrate is 3-Amino-9-ethylcarbazole (AEC). In other embodiments, the chromogenic substrate is 5-bromo-4-chloro-3-indolyl phosphate/tetranitroblue tetrazolium (BCIP/TNBT). In still other embodiments, the chromogenic substrate is Naphthol AS-MX phosphate+Fast Blue BB.

After treating the sample with the chromogenic substrate, the product is then counterstained for a period of time. Any counterstain that sufficiently contrasts the color of the chromogenic substrate may be used. A number of different counterstains are known to those skilled in the art, including, for example, methyl green and hematoxylin.

In some embodiments, the product is then counterstained for up to 8 minutes. In some embodiments, the product is counterstained for up to 4 minutes. In other embodiments, the product is counterstained for up to 2 minutes. In some embodiments, the product is counterstained for up to 1 minute. In some embodiments, the product is counterstained for up to 30 seconds. In some embodiments, the product is counterstained for up to 10 seconds.

In some embodiments, the counterstain is hematoxylin. Methods of using haematoxylin are known to those skilled in the art. See, e.g. Godwin Avwioro, Histochemical uses of Haematoxylin—A Review, JPCS Vol. 1, April-June 2011, 24-34. The concentration of hematoxylin generally ranges from about 1 g/L to about 2 g/L.

In some embodiments , the product is counterstained with hematoxylin for up to 8 minutes. In some embodiments, the product is counterstained with hematoxylin for up to 4 minutes. In other embodiments, the product is counterstained with hematoxylin for up to 2 minutes. In some embodiments, the product is counterstained with hematoxylin for up to 1 minute. In some embodiments, the product is counterstained with hematoxylin for up to 30 seconds. In some embodiments, the product is counterstained with hematoxylin for up to 10 seconds. In some embodiments, the product is counterstained with hematoxylin for between 10 seconds and 30 seconds.

In some aspects, the present disclosure is directed to a kit for use in immunohistochemical staining for GSK-3, wherein the kit comprises
(a) a GSK-3 inhibitor;
(b) a GSK-3 specific antibody;
(c) a secondary antibody that is specific for the GSK-3 specific antibody, wherein the secondary antibody also has a conjugated enzymatic activity;
(d) a chromogenic substrate for the enzymatic activity of the secondary antibody;
(e) a counterstain;
(f) and instructions for use of the components of the kit.

In some embodiments of the kits of the present invention, the GSK-3 inhibitor is 9-ING-41, tideglusib, LY2090314, CHIR-99021, CHIR-98014, SB216763, SB415286, AR-A014418, CG701338, or CG202796.

In some embodiments of the kits of the present invention, the GSK-3 inhibitor is 9-ING-41,

In some embodiments of the kits of the present invention, the GSK-3 specific antibody is a GSK-3beta specific monoclonal antibody. In other embodiments of the kits of the present invention, the GSK-3 specific antibody is a GSK-3α specific monoclonal antibody

In some embodiments of the kits of the present invention, the conjugated enzymatic activity is peroxidase activity or alkaline phosphatase activity. In some embodiments, the conjugated enzymatic activity is peroxidase activity. In other embodiments, the conjugated enzymatic activity is alkaline phosphatase activity.

In some embodiments of the kits of the present invention, the chromogenic substrate is diaminobenzidine (DAB), 3-Amino-9-ethylcarbazole (AEC), 5-bromo-4-chloro-3-indolyl phosphate/tetranitroblue tetrazolium (BCIP/TNBT), or Naphthol AS-MX phosphate+Fast Blue BB. In some embodiments of the kits of the present invention, the chromogenic substrate is diaminobenzidine (DAB),

In some embodiments of the kits of the present invention, the counterstain is methyl green or hematoxylin. In some embodiments, the counterstain is hematoxylin.

In some embodiments, the kits of the present invention also comprise instructions for use of the kit.

### EXAMPLES

Immunohistochemical studies were performed on formalin-fixed, paraffin-embedded sections of the breast, colon and pancreatic tumors in order to develop and optimize IHC staining procedure for detection of GSK-3β and GSK-3α expression in human tumor tissue.

### Materials and Methods

Formalin-fixed, paraffin-embedded sections of breast (5 cases), colon (2 cases) and pancreatic (2 cases) cancer were used. Tumor sections (5 μm) were deparaffinized, and antigen retrieval was carried out at 90-100° C. in citrate buffer for 10-40 min. The sections were incubated in 1% hydrogen peroxidase for 10 minutes to quench endogenous tissue peroxidase. Tumor sections were then incubated with primary GSK-3 isoform-specific antibody for 1 hour at room temperature. The primary GSK-3 isoform-specific antibodies used were as follows:

GSK-3beta (D5C5Z) XP rabbit mAb #12456, from Cell Signaling Technology

GSK-3alpha (D80D1) XP rabbit mAb #4818, from Cell Signaling Technology

The slides were stained using a standard EnVision+System-HRP kit (DAKO, Carpinteria, Calif.) according to the manufacture's protocol. Immunohistochemical reactions were developed with diaminobenzidine as the chromogenic peroxidase substrate, and slides were counterstained with hematoxylin. Negative control samples included replacement of the primary antibody with nonimmune IgG1 (Dako).

### Results

Using IHC staining of human tumor sections, the following dilutions of Ab were tested:

GSK-3α Ab 1:50 and 1:100

GSK-3β Ab 1:100, 1:200, 1:400

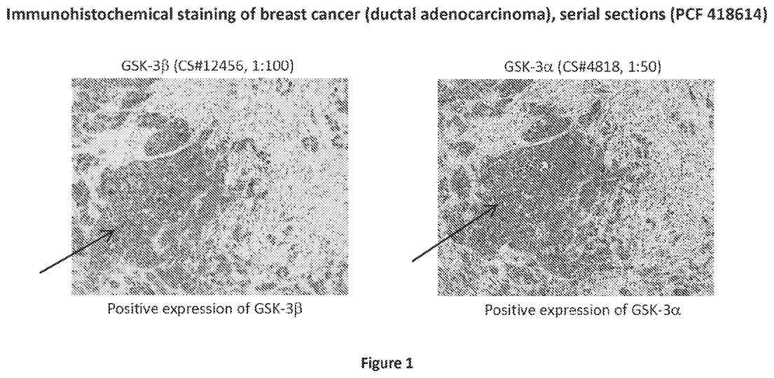

Specific staining of the target molecule with low background staining was observed in breast and colon tumor samples (FIG. 1, 2) at the following dilutions of Ab:

GSK-3α Ab 1:50

GSK-3β Ab 1:100

Tumor section of breast ductal adenocarcinoma PCF 418614 is recommended as positive control for IHC staining with antibodies against GSK-3α and GSK-3β at the dilutions as described above.

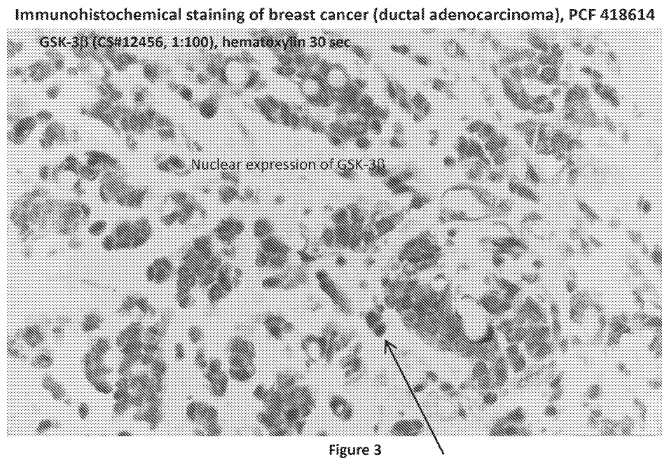

Eight minutes vs 30 seconds hematoxylin staining was tested with GSK-3β IHC staining using breast tumor sections (5 cases). Nuclear GSK-3β is better detectable in cancer cells after hematoxylin counterstaining for 30 seconds and therefore this time is recommended for hematoxylin counterstaining of GSK-3β-stained sections (FIG. 3, 4).

1. A method of treating a disease in a subject in need thereof, the method comprising: a) obtaining a tissue sample from the subject; b) measuring the expression level of a GSK-3 isoform in the sample, wherein the measuring is performed by immunohistochemical staining; c) comparing the expression level of the GSK-3 isoform in the tissue sample with the expression level of that GSK-3 isoform in a control sample; and d) administering an effective amount of an inhibitor of the GSK-3 isoform to the subject if an elevated expression level of the GSK-3 isoform in the tissue sample is detected when compared to the expression level of the GSK-3 isoform in the control sample.

2. The method of claim 1, wherein the expression level of GSK-3 isoform is nuclear expression.

3. The method of claim 1, wherein the expression level of GSK-3 isoform is cytoplasmic expression.

4. The method of claim 1, wherein the expression level of GSK-3 isoform is nuclear and cytoplasmic expression.

5. The method of claim 1, wherein the GSK-3 isoform is GSK-3β.

6. The method of claim 1, wherein the inhibitor of the GSK-3 isoform is 9-ING-41, tideglusib, or LY2090314.

7. The method of claim 6, wherein the inhibitor of the GSK-3 isoform is 9-ING-41.

8. The method of claim 1, wherein the GSK-3 isoform is GSK-3α.

9. The method of claim 1, wherein the disease is cancer.

10. The method of claim 9, wherein the tissue sample is a tumor sample.

11. The method of claim 9, wherein the cancer is breast cancer, brain cancer, esophagus cancer, stomach cancer, lung cancer, liver cancer, kidney cancer, thyroid gland cancer, spleen cancer, pancreas cancer, large bowel cancer, skin cancer, ovarian cancer, uterus cancer, prostate cancer, bladder cancer, testicular cancer, osteosarcoma, fibrosarcoma.

12. The method of claim 1, wherein the disease is a malignant lymphoproliferative disorder.

13. The method of claim 1, wherein the disease is pulmonary fibrosis or pleural fibrosis.

14. The method of claim 1, wherein the immunohistochemical staining comprises a step comprising counterstaining the tissue sample with a counterstain for about 30 seconds to about 8 minutes.

15. The method of claim 14, wherein the counterstain is hematoxylin and the counterstaining is for about 30 seconds.

16. An immunohistochemical method for detecting elevated expression of a GSK-3 isoform in a biological sample from an individual through an antigen-antibody reaction comprising, (1) contacting said biological sample with a first antibody specific for a GSK-3 isoform to give a antibody-contacted biological sample; (2) contacting said first antibody-contacted biological sample with a secondary antibody specific for said first antibody, wherein said secondary antibody has an enzymatic activity; (3) treating the product of (2) with a chromogenic substrate for said enzymatic activity; and (4) counterstaining the product of (3) with a counterstain for up to 8 minutes.

17. The method of claim 16, wherein the counterstain is hematoxylin.

18. The method of claim 17 wherein the counterstaining with hematoxylin is performed for up to 4 minutes.

19. The method of claim 17 wherein the counterstaining with hematoxylin is performed for up to 2 minutes.

20. The method of claim 17 wherein the counterstaining with hematoxylin is performed for up to 1 minute.

21. The method of claim 17 wherein the counterstaining with hematoxylin is performed for up to 30 seconds.

22. The method of claim 17 wherein the counterstaining with hematoxylin is performed for up to 10 seconds.

23. The method of claim 16, wherein the enzymatic activity of the secondary antibody is peroxidase activity.

24. The method of claim 16, wherein the enzymatic activity of the secondary antibody is alkaline phosphatase activity.

25. The method of claim 23, wherein the chromogenic substrate is a peroxidase substrate.

26. The method of claim 24, wherein the chromogenic substrate is an alkaline phosphatase substrate.

27. The method of claim 16, wherein the first antibody is a monoclonal antibody.

28. The method of claim 16, wherein the second antibody is a monoclonal antibody.

29. A kit for use in immunohistochemical staining for GSK-3, wherein said kit comprises
(a) a GSK-3 inhibitor;
(b) a GSK-3 specific antibody;
(c) a secondary antibody that is specific for the GSK-3 specific antibody, wherein the secondary antibody also has a conjugated enzymatic activity;
(d) a chromogenic substrate for the enzymatic activity of the secondary antibody;
(e) a counterstain;
(f) and instructions for use of the kit.

30. The kit of claim 29, wherein the GSK-3 inhibitor is 9-ING-41, tideglusib, LY2090314, CHIR-99021, CHIR-98014, SB216763, SB415286, AR-A014418, CG701338, or CG202796

31. The kit of claim 30, wherein the GSK-3 inhibitor is 9-ING-41.

32. The kit of claim 29, wherein the GSK-3 specific antibody is a GSK-3beta specific monoclonal antibody.

33. The kit of claim 29, wherein conjugated enzymatic activity is peroxidase activity or alkaline phosphatase activity.

34. The kit of claim 33, wherein conjugated enzymatic activity is peroxidase activity.

35. The kit of claim 29, wherein chromogenic substrate is diaminobenzidine (DAB), 3-Amino-9-ethylcarbazole (AEC), 5-bromo-4-chloro-3-indolyl phosphate/tetranitroblue tetrazolium (BCIP/TNBT), or Naphthol AS-MX phosphate+Fast Blue BB.

36. The kit of claim 35, wherein chromogenic substrate is diaminobenzidine (DAB).

37. The kit of claim 29, wherein the counterstain is hematoxylin or methyl green.

38. The kit of claim 37, wherein the counterstain is hematoxylin.

39. The kit according to claim 29, for use in a method of treating a disease in a subject in need thereof, the method comprising: a) obtaining a tissue sample from the subject; b) measuring the expression level of a GSK-3 isoform in the sample, wherein the measuring is performed by immunohistochemical staining; c) comparing the expression level of the GSK-3 isoform in the tissue sample with the expression level of that GSK-3 isoform in a control sample; and d) administering an effective amount of an inhibitor of the GSK-3 isoform to the subject if an elevated expression level of the GSK-3 isoform in the tissue sample is detected when compared to the expression level of the GSK-3 isoform in the control sample.
